(19) United States
(12) Patent Application Publication
(10) Pub. No.: US 2018/0178145 A1
(43) Pub. Date: Jun. 28, 2018

(54) INTEGRATED PROCESS FOR BIODIESEL PRODUCTION THROUGH THE APPLICATION OF SALINE DEMULSIFICATION, DISTILLATION AND ESTERIFICATION STAGES OF FATTY ACIDS AND DERIVATIVES

(71) Applicants: ECO 100 DESENVOLVIMENTO SUSTENTADO LTDA-ME, Rio de Janeiro (BR); COMPANHIA ESTADUAL DE AGUAS E ESGOTOS-CEDAE, Rio de Janeiro (BR)

(72) Inventors: Luciano Basto OLIVEIRA, Rio de Janeiro (BR); Hugo Villela DE MIRANDA, Rio de Janeiro (BR); Luiz Roberto Martins PEDROSO, Rio de Janeiro (BR)

(21) Appl. No.: 15/744,989

(22) PCT Filed: Jul. 11, 2016
(86) PCT No.: PCT/BR2016/050160

§ 371 Date: Jan. 15, 2018

(30) Foreign Application Priority Data
Jul. 16, 2015 (BR) BR 102015017003-3

(51) Int. Cl. B01D 17/04; C07C 67/08; C10L 1/02; C11C 3/04

(52) U.S. Cl. CPC B01D 17/045; C07C 67/08; C10L 2200/0476; C11C 3/04; C10L 2290/08; C10L 1/02

(57) ABSTRACT

The current invention refers to an integrated process for the production of biofuels, through the application of integrated saline demulsification, distillation and esterification stages for fatty acids and its derivatives in a process involving the breakdown of fatty acids stable emulsions and its derivatives through the isolated or joint utilization of physical processes, as well as saline demulsification of greasy residue denominated from aqueous emulsions, developed in such manner as to promote a suitable destination for such residue, such as the use for the production of biofuels, more specifically, biodiesel. The process proposed herein employs a raw-material purification methodology, which implies in better yields in the production of biodiesel through physical processes, such as sifting, filtration, and distillation of the extracted grease, followed by an esterification and transesterification process.

The current invention refers to an integrated process for the production of biofuels, through the application of integrated saline demulsification, distillation and esterification stages for fatty acids and its derivatives, more specifically a process involving the breakdown of fatty acids stable emulsions and its derivatives through the isolated or joint utilization of physical processes, as well as saline demulsification of greasy residue denominated from aqueous emulsions, developed in such manner as to promote a suitable destination for such residue, such as the use for the production of biofuels, more specifically, biodiesel. The process proposed herein employs a raw-material purification methodology, which implies in better yields in the production of biodiesel through physical processes, such as sifting, filtration, and distillation of the extracted grease, followed by an esterification and transesterification process.

### STATE OF THE ART

Greasy residue from grease trap boxes, from industrial processing waste, from agricultural processing waste, from urban processing waste, from animal fat and vegetal oil refining sludge are exposed in oil and fat for and respective degradation processes. The improper disposition of these residues in landfills and household sewage systems is a common practice (NETO et. al., 2000), being commonly found emulsified in grease trap boxes, which are also posteriorly discarded in Sewage Treatment Stations (Estações de Tratamento de Esgoto—ETE), collected by septic service trucks in commercial establishments or households.

Several processes known to the state of the art are related to the emulsion of fatty acids with the posterior esterification through acid catalysis in sight. Both triglycerides and fatty acids present a strong apolar character in its molecules and, for this reason, possess limited solubility in polar solvents, such as water, for instance. Furthermore, present a relatively high viscosity. Such characteristics make these compounds prone to form stable emulsions in water, especially fatty acids, which possess such high viscosities that present, on a vast majority, a paste, or even a solid form at room temperature. Thus, fatty acids aqueous emulsions have a solid aspect, retaining inside several types of substances, such as, for example, plastics and paper amongst others, given that such are available in the surrounding area, as is the case for grease trap boxes and sewage scum.

As the presence of water inhibits the esterification reaction, once such is a reagent in the opposite reaction, which is in reversible equilibrium with the esterification reaction, breaking down the aqueous emulsion is indispensable for the desired process to occur. This break down has been performed by the extraction of grease matter with an apolar solvent immiscible in water, but with low viscosity. Thus, the resulting apolar solution also presents low viscosity, which allows for coalescence of water drops dispersed in the emulsion and the consequent stage separation: a phase rich in fatty acids dissolved in the apolar solvent, in the top part, and another denser aqueous phase in the lower part.

These emulsions are colloidal dispersions, composed by a dispersing phase and a dispersed phase. The emulsions receive a classification in accordance to the particles size, which vary from 0.02 to 0.2 micron, where the disperse phase may be solid, liquid or gaseous: the same is considered for the dispersing phase (SOUZA, 2003).

However, all techniques applied industrially utilize extraction with a solvent to separate the fatty acids in the aqueous phase. Thus, the state of the art verified that some patents related to the demulsification use other methods, reagents and intended use, differently from fatty acids, mainly residue, as for example, patent PI 0008561-8, that describes the use of phenolic resin and acrylic acid to demulsify hydrocarbon and water. The patent request PI 1000113-1 describes the acid demulsification methodology, which creates unpleasant small and requires proper equipment to remove such odors, and in addition, strong acids may compromise the organic raw-material promoting chemical and physical alterations in such. Patent PI 0603824-7 describes a methodology using organic solvent and lipase to break down such emulsions.

Although it is a method to separate grease material from water, the use of the extraction using a solvent entails a series of problems for the process as a whole. The first one is related to the difficulty in recovering the grease material, once according to Raoult's law, the boiling point of the binary mixture still containing part of the volatile solvent is very close to the fatty acids boiling point, and in some cases, above 350° Celsius. This temperature is sufficient for the occurrence of several undesired collateral reactions; furthermore, represents a high energy consumption for the recovery process. Another important issue that deserves to be cited is related to the capture of a portion of the solvent in the aqueous phase, due to the presence of emulsifying substances that are naturally present in oil and are added on purpose in some cases, such as in household sinks, in the form of detergents and soap to facilitate carrying the grease matter in aqueous phase. These substances have the capacity to generate dispersed apolar micelle in the polar phase. This fact makes it very difficult to recover the solvent in the aqueous phase.

Another important problem in extraction with a volatile solvent is related to environmental effects, which two of such deserve to be mentioned: the condition of water pollutants and evaporation. The first refers to the fact that organic solvents are classified as water pollutants. This renders the final disposal of the aqueous waste from the process problematic, as the same, as already mentioned, is contaminated with organic solvent, usually hexane. The other effect refers to the loss of solvent to the atmosphere, given that such solvent contributes to the “photochemical smog” process, precisely one of biodiesel weaknesses, once several studies have related biodiesel usage, a fuel source that can be obtained from fatty acids esterification, with the increase of Nox emissions, the other reagent necessary for the “photochemical smog” process to occur and which consequences are harmful to the metropolis environment.

It is also worth to mention that the utilization of inflammable solvents increases the concern with fire hazards and explosions, as well as generating the need to make investments in fire prevention and fire-fighting, in case of any occurrence.

Once the raw-material is demulsified, that is, the grease matter is extracted from the aqueous emulsion, or obtained from thermal, enzymatic hydrolysis or by chemical catalysis of mono-, di- and triglycerides, or from the sewage sludge, grease trap boxes, industrial processing residue, agricultural processing residue, urban processing residue, of animal fat such as, bovine, porcine, caprine, ovine and equine tallow, fish oil and chicken fat, duck and turkey and refining sludge of new or used crude or refined vegetable oils such as soybeans, canola, canola, palm, peanut, sunflower, cotton, olive, coconut, babassu, canola, corn, mammoth, macaúba and jatropha, it becomes necessary to purify the same this raw-material.

Alcoholysis or esterification of waste oils and fats is totally dependent on the quality of the material at hand. Greasy matter, such as soybean, canola, rapeseed, palm, palm, peanut, sunflower, cotton, olive, coconut, babassu, canola, corn, mammal, macauba and jatropha, repeatedly used in frying processes or from sewage sludge, fat, industrial processing tailings, agricultural processing tailings, urban processing tailings, animal fat such as beef tallow, swine, goat, sheep and equine, fish oil and chicken fat, duck and turkey and refining lees of new or used vegetable oils, raw or refined, undergoes degradation by hydrolytic and oxidative reactions, accelerated by the high temperature of the process, or by bacteria, which are mainly responsible for the modification of the physicochemical and organoleptic characteristics of oils. There is an increase in the viscosity, acidity and rancidity, generating unpleasant odors. The biodiesel quality directly depends upon these oils and fats, given that the impurities contained in such have a strong influence over the production process efficiency.

One of the aspects from the present invention involves the purification method for the raw-materials used, in other words, in the improvement of the raw-material at hand. After the demulsification proposed in this invention of fatty materials such as soybean, canola, rape, palm, palm, peanut, sunflower, cotton, olive, coconut, babassu, canola, corn, mammoth, macauba and jatropha, repeatedly used in processes or from sewage sludge, grease trap boxes, industrial processing waste, agricultural processing waste, urban processing waste, animal fat such as beef tallow, swine, goat, sheep and equine, fish oil and chicken, duck and turkey fat and refining sludge of new or used crude or refined vegetable oils which have been degraded by hydrolytic and oxidative reactions accelerated by the high temperature of the process or by bacteria and which were separated from the aqueous phase, the fatty acids obtained are distilled at temperatures ranging from 100° C. to 350° C. and a vacuum between 300 mmHg and 700 mmHg. As the grease material is composed of several molecules of different sizes, that is, carbon chains having from 6 to 24 carbons, distillation is conducted with varying temperatures starting at about 100° C. and ending at about 350° C., initiating the distillation by the fatty acids with lower carbon chain. The vacuum is more effective when the work is performed between 500 mmHg and 600 mmHg.

Once the raw material is prepared, that is, the grease is extracted from the aqueous emulsion, or obtained from the thermal, enzymatic hydrolysis or by chemical catalysis of mono-, di- and triglycerides and separated from other contaminants such as plastics and paper, through physical processes, such as sieving, filtration and distillation, the esterification itself can be performed. Esterification by acid catalysis employs temperatures typically between 60° and 90° C. under agitation with a reaction time between 3 and 7 hours. In addition, the use of excess alcohol, which in some patent applications reaches a ratio of up to 10/1 as for Patent PI 0500333-4, is being adopted as a manner to improve the conversion of the reactants into the product, or even more like in patent application PI 0301254-9, which advocates up to 15/1 as an efficient way to carry out the conversion. However, even against all this stoichiometric excess of alcohol, the ester conversion in patent application PI 0301254-9 is about 60%. This percentage is well below that expressed in the ANP Resolution 007/2008 which now requires a minimum ester content of 96.5% so that the fuel can be classified as biodiesel in Brazil.

Besides not guaranteeing the specification for biodiesel as required in Brazil, the adoption of a extremely high alcohol excess in the route by acid catalysis leads to several inconveniences in the process. The first one refers to the need for reactors large enough to contain the entire reaction mass, which causes an increase in equipment costs. Another drawback that may be mentioned is related to the increase in energy consumption to heat up the reaction mass to the reaction temperature, as well as the additional energy required for the recovery of the excess alcohol, which is effected by means of distillation. It is worth mentioning that the adoption of alcohol in excess is justified by two factors. One is that at the time of the patent submission mentioned above, there was no specification as to the minimum ester content in the fuel, so that it could be classified as biodiesel, which only occurred in 2008. The other factor refers to the fact that this is the most common method to increase conversion without the need to withdraw water from the reaction medium, which is formed with the development of the reaction itself and tends to reverse the process towards the reactants formation.

In the process proposed by the present invention, that is, an integrated process between the demulsification of the grease material, purification and esterification of the raw-material, ester contents were in the order of 97% using a short-chain alcohol with a rate of 0.5/1, demonstrating the effectiveness of the process.

### INVENTION SUMMARY

The present invention has as objective the utilization of grease matter with over five carbon atoms and a high acidity index, whether obtained naturally, whether produced from the glycerides hydrolysis, in esterification reactions with short chain alcohols, or more specifically, with up to five carbon atoms, such as methanol, ethanol, isopropanol and tert-butanol, by acid catalysis without the use of inflammable organic solvents, harmful to health and the environment in the extraction of the raw material. Instead, the grease material is made available for the esterification process by breaking down the aqueous emulsion by the joint or isolated use of physical demulsification processes such as microwave (frequency between 2.4 GHz and 2.5 GHz), ultrasound (frequency between 25 KHz and 10 MHz), electric field application with continuous current or alternating current pulses of up to 10 KHz and voltage gradient up to 10 KV/cm, heating with vigorous stirring, use of centrifuges to accelerate phase separation, among others, or chemical process to reducing the pH of the reaction medium using the acid catalyst itself from the esterification reaction, which may be residual therefrom, which has the reaction capacity to break the emulsion, even if aided by other separation processes from the emulsified phases, such as the saline demulsification using electrolyte solution, more specifically sodium chloride (NaCl). This is a more economically competitive and more environmentally friendly procedure.

### DESCRIPTION OF THE INVENTION

The first step of the present invention consists in the separation of the grease matter from other contaminants with the intent of making it suitable for the esterification process. Some grease matter sources, such as those from household grease trap boxes or the primary decanters of sewage treatment plants, have an acid content of 30% to 90%; others as refining sludge from vegetable oils have typical contents in the order of 30% to 70%. The increase in the acid content is related to the natural degradation process of the glycerides in fatty acids. Thus, the more degraded the raw material, the higher the acidity content. Among the mechanisms that lead to this degradation can be mentioned oxidative rancidity, related to the reaction with oxygen in the air, and hydrolytic rancidity, related to enzymes that degrade glyceride in aqueous medium. The higher the acidity content, the higher the viscosity of the grease material and, therefore, the higher the propensity to compose stable emulsions with polar solvents, especially water. Thus, it is common to form water emulsions and grease with water content, varying between 40% and 80%, with a solid appearance and imprisoning in its interior other materials such as pieces of wood, paper, plastics, even objects of fairly high density as sand, small stones and metal. Under these conditions, the emulsion is not suitable for use in the esterification process and, therefore, it is indispensable to separate such contaminants grease matter.

The stability of these emulsions is mainly due to two factors. The first refers to the grease material high viscosity, which increases appreciably with the proportion of fatty acids present. The second factor relates to the occurrence of emulsifying substances that act at the water interface with the grease matter. These emulsifiers are naturally present in vegetable oils and even in petroleum, and in addition, are added on purpose in some cases, such as in kitchen sinks to facilitate the transport of grease matter. Basically, the emulsifiers have in their molecule a polar part, which presents affinity with water, and an apolar part, with affinity with the grease phase. With this, the emulsifiers form micelles by isolating the dispersed phase from the dispersing phase.

Thus, one way of breaking the stability of grease emulsions with water is to heat the emulsion in agitation until the grease material viscosity is reduced to allow movement of water particles which are combining to form new particles, and the phase separation may occur. However, heating alone is often not enough to break the emulsion, due to the presence of the emulsifying agents. A bond with an ionic character between a carbon atom and a strongly electropositive chemical element generally forms the polar part of the emulsifying agent. The action of the electrolyte on the demulsification is to reduce the thickness of the double layer. Its purpose is achieved every time the electrostatic colloidal protection is broken or reduced. With the reduction of this double layer, the particle becomes more susceptible to shocks by the action of Brownian motion, also favored by agitation, thus occurring coalescence and, consequently, the demulsification. The colloidal particle, upon movement within the fluid, undergoes a shear in relation to this fluid. This shear force is unable to pull off some layers of the fluid itself or the protective film, in this case, formed by the detergent, which covers the particle. A medium rich in salts reduces or eliminates the thickness of the double layer, so that simple aggregation by collision between the particles is easy. Even if there is a surface charge due to the protective film, it will not be sufficient to avoid the collision caused by the Brownian motion, also favored by mechanical agitation, causing particles to arrive very close to each other and, with the reduction of the double layer, the of van der Waals forces (forces of attraction) will overcome the coulombian forces (charges of the same sign), giving room for the demulsification to occur.

The result of this reaction is the formation of a generally soluble ionic salt layer and an insoluble nonpolar compound, both in the aqueous medium and incapable of forming micelles. Such conditions, i.e., high enough temperature to reduce the viscosity of the grease material and salt availability to break the double layer, are sufficient to depolarize the emulsifier and the phase separation may occur.

The temperature required to allow the coalescence of droplets depends on the specific composition of the grease matter at hand. However, the values are often in the range from 60° to 90° Celsius, preferably around 70° C. Likewise, the amount of salt (sodium chloride—NaCl) required to depolarize the demulsifying agents depends on the concentration thereof, often this value ranges between 5% and 15% by the emulsion weight, preferably around 10%.

Once the heating and addition of the salt are done, the coalescence of droplets of the dispersed phase and the consequent separation of the phases begins to occur. At this point, the emulsion should decant for a period ranging from one to twenty four hours, at a room temperature ranging between 60° to 90° Celsius, preferably about three hours. The settling time will depend on the viscosity, the average density of the grease material in separation and the utilization degree intended to be achieved in the decantation process. As a way to accelerate the separation of the phases, a centrifuge may be used. At this point, it is possible to sieve the impurities of the grease phase. The mesh of the sieve will depend on the average size of the impurities, since they vary greatly depending on the origin of the raw material used. However, it has been found that the uses of filters complicates the process in such manner, without leading to an improvement in the final yield, and are therefore totally expendable. Likewise, the use of crushers or mills in a step prior to the breaking of the emulsion did not lead to an improvement in the process. The demulsification can be performed continuously or in batch. In the first case, centrifuges attached to sieves with appropriate mesh sizes to the size of the contaminants present in the specific raw material should be used. In the batch process, the best way to promote the phase separation is by pouring the grease material through a spillway in the upper part of the vessel by increasing the internal pressure of the equipment. This can be achieved by injecting water under pressure, or by expanding a plunger or any other equipment that allows for or increases the volume of compressed air within the demulsifying vessel without, however, letting the air escape through the upper part, where it is desired to pour the demulsified grease. With this method, it is possible to increase the internal pressure of the vessel, thus expelling the raw material through the spillway, at the same time that it is sieved under pressure. Another way to separate grease material from the remaining impurities is to use suction pumps, transferring the grease material to the next purification step.

Acid demulsification usually releases unpleasant odors, a problem that can be mitigated by the use of tall chimneys, or the passage of gases exuded by deodorizing filters or by any other deodorization method available, in which cases the operating costs would be higher in comparison to the construction of high chimneys that require an increase in investment.

Saline demulsification, the core aspect of the present invention, does not require using resources such as chimneys or deodorizers, since such are used in the acid demulsification, for the dispersion of foul and undesirable gases, but are transferred to another vessel in the course of purification by a vacuum pump, suction pump or by any other methodology described in this invention.

Fatty acids are carboxylic acids with a long aliphatic chain. These are often classified by the absence or presence of unsaturation. Acids containing two or more unsaturation are called polyunsaturated and represented by numerical symbols such as, for example, C 18: 2, which represents linoleic acid, the first number juxtaposed to the symbol C indicates the number of carbon atoms and the second number, the amount of double bonds.

Distillation is a separation method based on the liquid-vapor equilibrium phenomenon between mixtures. In practical terms, when two or more substances form a homogeneous liquid mixture, distillation may be a method to separate these substances. These substances just have to have reasonably different volatilities (i.e., the respective boiling points are relatively far apart). It is also possible to separate a volatile liquid from a non-volatile solid. The use of distillation as a separation method is widely used by the modern chemical industry. It can be found in almost all industrial chemical processes in the liquid phase where purification is required.

Knowledge of the physical and chemical properties of fatty acids is one of the prerequisites for industrial production and technical applications. Thermodynamic and transport properties are required for the calculation of heat transfer, for separation processes by distillation and chemical reactions.

The fusion behavior is very specific for each fatty acid. The fatty acids fusion point depends on the number of carbons, the degree of saturation and the chain structure. In a linear chain, the fatty acids fusion point increases with the increase of the chain. For unsaturated fatty acids, the behavior is a little more complex.

The heat from vaporization and the properties related to the boiling point and vapor pressure are very important for fatty acids distillation and thermal separation by means of fractional distillation. To avoid decomposition, the thermal processes are performed at the lowest temperature possible under vacuum (ULLMAN, 2003). The distillation of the grease material starts at temperatures around 100° C., when the fatty acids from lower carbon chains are vaporized and extracted, up to temperatures of 350° C. for carbon chains considered large. The vacuum directly influences the distillation temperature. The higher the vacuum, the lower the temperature used, ranging from −300 mmHg to −700 mmHg, preferably using values between −500 mmHg and −600 mmHg.

Although studies and researches related to fatty acids distillation are well known, no prior state of the art document, especially patent or patent application, uses waste grease such as sewage slag, grease traps boxes, industrial processing, agricultural processing waste, urban processing waste, animal fat such as bovine tallow, swine, goat, sheep and equine, fish oil and chicken fat, duck and turkey and oil refining lees raw or refined, such as soybeans, canola, rapeseed, palm, palm oil, peanut, sunflower, cotton, olive, coconut, babassu, canola, corn, mammoth, macauba and jatropha, in order to purify the grease matter. Through the distillation process, all impurities contained in the grease material are eliminated, thus obtaining good quality raw material to be transformed into biofuel.

Since the fatty material is purified and separated from its contaminants, the esterification is carried out as demonstrated below. Once the fatty matter is distilled, it is basically composed of fatty acids and, consequently, the acidity is very high, around 100%, not requiring hydrolysis processes, which would serve for the transformation of mono-, di-, and triglycerides into fatty acids.

The concern about the quality of the raw material is reflected in the biodiesel production through esterification, as less quantities of reagents are needed. In order to improve the reactants conversion into the product, the use of excess alcohol, although in some prior state of the art patent applications, it reaches a ratio of up to 10/1, as is the case of patent application 0500333-4, or even more, as is the case of patent application PI 0301254-9, which advocates up to 15/1 as an efficient form to convert, whereas in the present invention, proportions range between 0.5/1 to 2/1 alcohol/fat, and are sufficient for the total conversion of fatty acids into biodiesel.

Esterification is reached by the reaction of fatty acids with short chain alcohol, preferably one to five carbons, such as methanol, ethanol, n-propanol, n-butanol, sec-butanol, iso-butanol among others in the presence of homogeneous acid catalysts such as sulfuric acid, hydrochloric acid, phosphoric acid, sulfonic acid, methanesulfonic acid and toluenesulfonic acid, among others, or heterogeneous substances that have the thermal stability and acidity of Bronsted and/or Lews in the conditions of such as calcium chloride, ferric chloride, zinc chloride, ferric sulfate, sulfated zirconia, nihobic acid and zeolites, provided they have hydrogen as the exchange cation, in the proportion of 0.5% to 10% %, preferably between 2% and 6%.

The reaction temperature is the boiling temperature of the mixture between reactants, between 60° C. and 90° Celsius, preferably at temperatures between 70° C. and 80° Celsius. The reaction time varies from one hour to seven hours, preferably from two hours to five hours.

At the end of the reaction the product is washed and transferred to transesterification in the proportions between 0.2/1 and 1/1 alcohol/ester and an alkaline catalyst, such as sodium hydroxide, potassium hydroxide, among others, and washed again with cold water in proportions between 5% and 30%, preferably in proportions between 10% and 20%. The biofuel is then washed and dried.

1. An integrated process for the production of biodiesel by applying the steps of salt demulsification, distillation and esterification of fatty acids and respective derivatives, characterized in that it comprises a first stage of demulsification for the emulsions separation phase containing water and fatty acids, followed by a purifying step of the fatty matter by a physical process and by an esterification step of the fatty acids.

2. A process in accordance to claim 1, characterized in that the fatty acid is produced from sewage slag, grease traps boxes, industrial processing waste, agricultural processing waste, urban processing waste, animal fat or refining lees of vegetable oils.

3. Process in accordance to claim 1, characterized in that it is the fatty acid obtained from the thermal, enzymatic hydrolysis or by chemical catalysis of mono-, di- and triglycerides from new or used, crude or refined vegetable oils.

4. Process in accordance to claim 3, characterized in that it is the vegetable oil of soybean, canola, rapeseed, palm, palm, peanut, sunflower, cotton, olive, coconut, babassu, canola, corn, mammoth, macauba or jatropha.

5. Process in accordance to claim 1, characterized in that it is the fatty acid obtained from the thermal, enzymatic hydrolysis or by chemical catalysis of mono-, di- and triglycerides derived from animal fat.

6. Process in accordance to claim 5, characterized for animal fat from bovine, porcine, caprine, ovine and equine tallow, fish oil and fat of chicken, duck or turkey.

7. Process in accordance to claim 1, characterized by a fatty acid with over 5 carbon atoms.

8. Process in accordance to claim 1, characterized by the de-emulsification carried out at temperatures ranging from 60° to 90° Celsius.

9. Process according to claim 8, characterized in that the de-emulsification is preferably carried out at a temperature of 70° C.

10. Process according to claim 1, characterized in that the de- emulsification is carried out with the addition of salt in an amount, which varies between 5% and 15%, by weight of the emulsion mass.

11. Process according to claim 10, characterized in that the de-emulsification is preferably carried out with the addition of salt for 10% by weight of the emulsion mass.

12. Process according to claims 10, characterized in that the salt is sodium chloride.

13. A process in accordance to claim 1, characterized in that the de-emulsification is conducted continuously.

14. A process in accordance to claim 13, characterized by the use of centrifuges coupled to sieves to accelerate the separation of the phases after the settling step.

15. Process according to claim 1, characterized in that the demulsification is carried out in batches.

16. A process in accordance to claim 15, characterized by the use of a spillway at the top of the vessel to promote separation of the phases after the settling step by increasing the internal pressure of the vessel.

17. A process in accordance to claim 14 or 16, characterized by the use of suction pumps to promote phase separation after the decantation step by transferring the grease material to the next purification step.

18. A process in accordance to claim 1, characterized by the purification of the grease material by a distillation process prior to the esterification process operating with a gradual rise in temperature between 100° C. and 350° C.

19. A process in accordance to claim 1, characterized by the purification of the grease material by a distillation process prior to the esterification process operating under vacuum between −350 mmHg and −700 mmHg.

20. A process in accordance to claim 19, characterized by the purification of the grease material by a distillation process prior to the esterification process, preferably with a vacuum operating between −500 mmHg and −600 mmHg.

21. A process in accordance to claim 1, characterized by the use in the esterification step of the fatty acids having an alcohol/fat ratio of between 0.5/1 and 2/1 with respect to the fat to be esterified.

22. A process in accordance to claim 1, characterized in that it preferably employs 0.5/1 in relation to the fat to be esterified.

23. Process according to claims 21, characterized in that the esterification is by acid catalysis followed by transesterification.

24. A process in accordance to claim 1, characterized in that it uses an amount of catalyst ranging from 0.5% to 10% of the mass of the material to be esterified.

25. Process according to claim 24, characterized in that it preferably uses an amount of catalyst ranging from 2% to 6% of the mass of the material to be esterified.

26. A process in accordance to claim 1, characterized in that it is carried out by homogeneous catalysis.

27. A process in accordance to claim 1, characterized by the use in the esterification of lower alcohol, preferably having from one to five carbon atoms.

28. A process in accordance to claim 1, characterized in that the reaction temperature in the esterification ranges from 60° C. to 90° C.

29. A process in accordance to claim 28, characterized in that the reaction temperature in the esterification is preferably between 70° C. and 80° C.

30. Process according to claim 1, characterized in that the time of the esterification reaction is from 1 hour to 7 hours.

31. A process in accordance to claim 30, wherein the time of the esterification reaction is preferably from 2 hours to 5 hours.

32. A process in accordance to claim 1, characterized in that after the completion of the esterification reaction the product is washed and conducted to the step of transesterification at the ratio of alcohol/ester between 0.2/1 and 1/1, in presence of alkaline catalyst to obtain the biofuel.
